(19) United States
(12) Patent Application Publication
(10) Pub. No.: US 2013/0296813 A2
(43) Pub. Date: Nov. 7, 2013

(54) SNAP-ON TWO-PIECE EMERGENCE PROFILE HEALING ABUTMENT

(71) Applicant: Sang-Hoon Park, Arlington, VA (US)

(72) Inventor: Sang-Hoon Park, Arlington, VA (US)

(73) Assignee: University of Maryland, Baltimore, Baltimore, MD (US)

(21) Appl. No.: 13/375,418

(22) PCT Filed: May 28, 2010
(86) PCT No.: PCT/US2010/036540

§ 371 Date: Nov. 30, 2011

Related U.S. Application Data
(60) Provisional application No. 61/182,810, filed on Jun. 1, 2009.

(51) Int. Cl. A61F 5/442; A61F 5/449; A61F 5/44

(52) U.S. Cl. 604/318; 604/317; 604/345; 604/334

(57) ABSTRACT

A snap-on two-piece emergence profile healing abutment provides a trans-gingival piece that can create various gingival shapes and contours of the dental implant emergence profile. The device consists of two main components: a central piece similar to standard healing abutments, having multiple facets; and an interchangeable outer piece used as a gingival emergence profile shell. The gingival emergence profile shell can be configured in various sizes and shapes to best estimate the size and shape of a dental crown to be restored.

### TECHNICAL FIELD

The present invention relates generally the field of dental implants, and more particularly the invention relates to a novel healing abutment having a snap-on emergence profile shell.

### BACKGROUND ART

Implant dentistry involves the restoration of one or more teeth in a patient's mouth using artificial components. Such artificial components typically include a dental implant and a prosthetic tooth and/or a final abutment that is secured to the dental implant. The use of dental implants has become increasingly popular since their commercial introduction. Initial attention of implant restoration of the mouth was focused on the achievement of sound integration of a permanent fixture to the surrounding bone. Success was determined by the degree of osseointegration, the bonding between the bone and implant to firmly anchor the implant fixture in place in order to provide a secure base for the prosthesis. Nowadays, dental restorations are frequently coupled with titanium implants placed in a patient's bone structure underlying missing teeth for the purpose of replacing the lost teeth. A transmucosal abutment is connected to the implant fixture, which thereby emerges from the patient's soft tissue or gum and provides the structure upon which to mount the restoration. A restoration, shaped like a tooth, is typically connected to the abutment by either cement or a screw.

As the dental community relied more and more on this field of prosthodontics, a variety of implants and appliances became available. However, the concentration on providing biocompatible fixtures in the mouth resulted in inadequate attention being paid to providing restorations that are aesthetic and integrate fully, and are more compatible to the patient's gums and gum line.

One of the problems encountered in this approach was the establishment of an emergence profile of the implant-supported restoration that supported the gingival tissue to give it a natural appearance. One of the reasons for this problem was that the manufactured healing abutments placed on the implant were typically cylindrical in shape rather than being the size and shape of the root of the tooth that was extracted. In order to fabricate an interim restoration that would have the correct anatomic root shape, the clinician was required to spend a significant amount of time building the root form from scratch for each implant-supported restoration.

Traditionally, prior to the dental implant impression step, a cylindrical uniform shaped healing abutment is inserted to shape the gingival emergence profile for aesthetic outcome. Depending on the gingival contour at the time of making an impression, the shape of the final crown will be significantly affected. Unfortunately, teeth vary in size and shapes. Hence, this becomes a serious clinical dilemma for aesthetic reasons. To resolve this problem, various companies have come up with standard healing abutments with different diameters. However, this only solves the size issues but does not solve the issues of shape of the gingival profile. Dentists, therefore, usually make a provisional/temporary crown by chair-side to overcome the shape issue. However, this requires several additional months for complete healing and gingival profile shaping.

Examples of healing abutments for dental implant systems are shown in U.S. Patent Application Publication No. 2008/0206709 and in U.S. Pat. Nos. 5,073,111; 5,417,568; 6,129,548; and 6,431,866. The dental prosthesis creates gingival contours surrounding an implant prosthesis, which imitate the gingival contours around natural teeth as well as around conventional fixed prosthodontics restorations.

### DISCLOSURE OF INVENTION

None of the prior art discloses an emergence profile shell that is interchangeable and can snap-on to a fixation device. The Snap-On Two-Piece Emergence Profile Healing Abutment of the present invention addresses/solves (1) clinical time issue, (2) size, and (3) shape. This has several advantages. Firstly, since the clinical technique is not so different from what the clinicians do with the currently existing products, the clinician can adopt this piece very easily (ease of use). Secondly, due to the presence of pre-fabricated gingival profile shells manufactured for the average tooth shape and size at various locations in the mouth, this will save tremendous clinical chair time (easy adaptation). Thirdly, since this can be done at the time of the surgical implant placement, this saves months of healing time (reduced gingival forming time). Lastly, the clinical chair time will be reduced without compromising the clinical outcome.

Accordingly, it is an object of the present invention to provide an emergence profile shell that avoids the disadvantages of the prior art.

It is another object of the present invention to provide healing abutments with various shaped top surfaces, which can provide a better match with the various shaped tooth analogues. A related object of the present invention is to facilitate tissue manipulation during crown fabrication procedures. A further related object is to develop a proper emergence profile.

A further object of the present invention is to provide healing abutments that create gingival contours surrounding an implant prosthesis, which imitate the gingival contours around natural teeth as well as around conventional fixed prosthodontics restorations.

It is another object of the present invention to provide an emergence profile shell that establishes a natural emergence profile and supports the gingival tissue in a practical and cost-effective manner. A related object of the present invention is to establish gingival health, contour, and aesthetics similar to conventional crown restoration.

A further object of the present invention is to provide healing abutments designed to prevent unnecessary damage or delay to the healing of the gingival tissues.

These and other objects of the present invention are accomplished by enabling a snap-on two-piece emergence profile healing abutment having a trans-gingival piece that can create various gingival shapes and contours of the dental implant emergence profile. The device consists of two main components: a central piece similar to standard healing abutments, having multiple surface facets; and an interchangeable outer piece used as a gingival emergence profile shell. The gingival emergence profile shell can be configured in various sizes and shapes to best estimate the size and shape of a dental crown to be restored.

### BRIEF DESCRIPTION OF THE DRAWINGS

The above and other features, aspects, and advantages of the present invention are considered in more detail, in relation to the following description of embodiments thereof shown in the accompanying drawings, in which:

FIG. 1 is a top plan view of a pre-made gingival emergence profile shell according to an embodiment of the present invention.

FIG. 2 is a side elevational view of a pre-made gingival emergence profile shell according to an embodiment of the present invention.

FIG. 3 is a top plan view of a customizable gingival emergence profile shell according to an embodiment of the present invention.

FIG. 4 is a side elevational view of a customizable gingival emergence profile shell according to an embodiment of the present invention.

FIG. 5 shows a shell removal tool according to an embodiment of the present invention.

### BEST MODE(S) FOR CARRYING OUT THE INVENTION

The invention provides an anatomical restoration dental implant system that is attachable to an anatomical restoration dental implant fixture embedded into a patient's jawbone. The Snap-On Two-Piece Emergence Profile Healing Abutment of the present invention is a trans-gingival piece that can create various gingival shapes and contours of the dental implant emergence profile. The invention consists of two main components: (1) a central piece: similar to a standard healing abutment having multiple surface facets and (2) an interchangeable outer piece: a gingival emergence profile shell. In a preferred embodiment, the gingival emergence profile shell will come in various sizes and shapes to best estimate the size and shape of the crown to be restored.

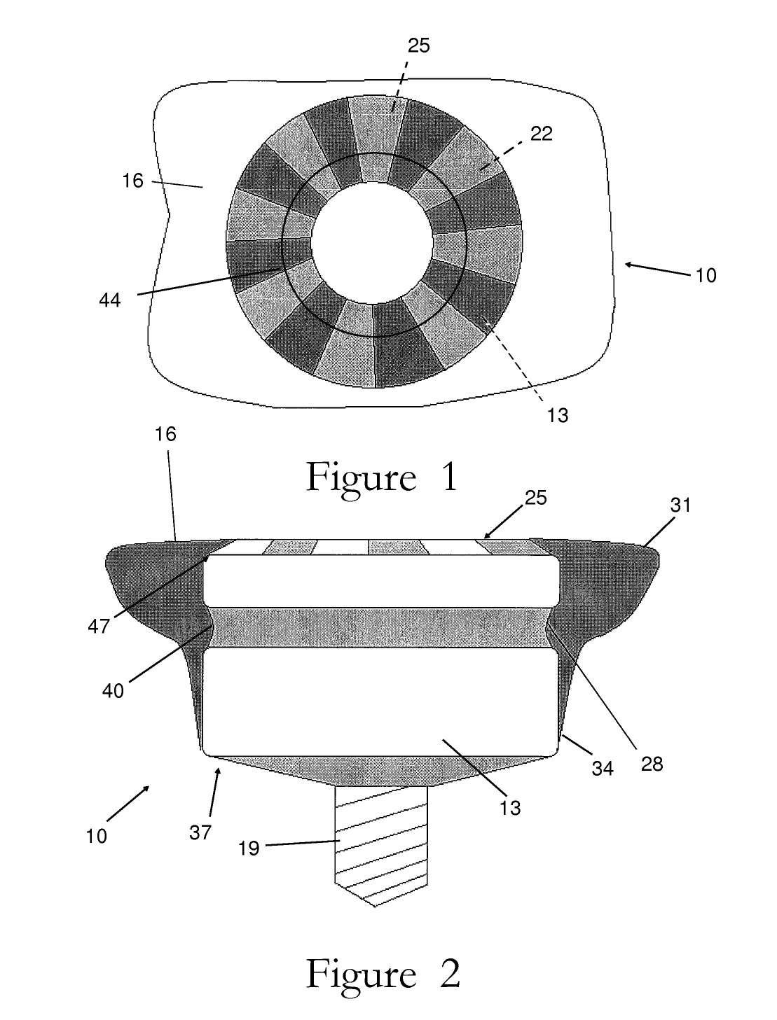

Referring to the drawings, FIGS. 1 and 2 show a pre-made healing abutment, indicated generally as 10, according to the present invention. The pre-made healing abutment 10 includes a central abutment piece 13 and a gingival emergence profile shell 16. The abutment piece 13 is generally cylindrical in shape and adapted for attachment to an oral implant, typically by using a screw or threaded post 19. The top surface 22 of the abutment piece 13 includes a plurality of facets 25 that create a multi-faceted occlusal stop/lock. In some embodiments, a plurality of teeth or other features that will lock the orientation of the device can be used. A detent 28 is formed around the periphery of the cylindrical wall of the abutment piece 13.

The profile shell 16 comes in a variety of sizes and shapes corresponding to an average shape of a tooth in various locations in the mouth. In a preferred embodiment, the profile shell 16 should be made of a compatible material having sufficient strength and flexibility. Additionally, the pre-made profile shell 16 is normally wider in a top region 31 and tapered toward the bottom 34. The profile shell 16 includes a hollow, cylindrical central area 37 and is adapted to be removably placed over the abutment piece 13. A lip 40 is formed on an inner wall of the profile shell 16. The lip 40 is sized and configured to engage the detent 28 of the abutment piece 13 in a “snap” engagement when the pre-made shell 16 is fit into place on the abutment piece 13. A top rim 44 of the shell 16 overlaps at least a portion of the top surface 22 of the abutment piece 13. The inner surface 47 of the top rim 44 includes a plurality of facets that are configured to match the facets 25 of the top surface 22 of the abutment piece 13 in order to create a multi-faceted occlusal stop/lock.

In use, the abutment piece 13 will be secured to an underlying dental implant by the screw or threaded post 19. A clinician will then select a pre-made gingival emergence profile shell 16 from an “Emergence Profile” guide. The pre-made profile shell 16 is aligned with the surrounding gum line and teeth for flexible orientation of the pre-made gingival profile shell 16 and then snapped onto the abutment piece 13. The plurality of multiple facets 25 on the top surface 22 of the abutment piece 13 when matched with the facets on the inner surface 47 of the top rim 44 prevents rotation of the emergence profile shell 16. The design of the present invention enables different emergence profile shells 16 to be interchangeable on the abutment piece 13.

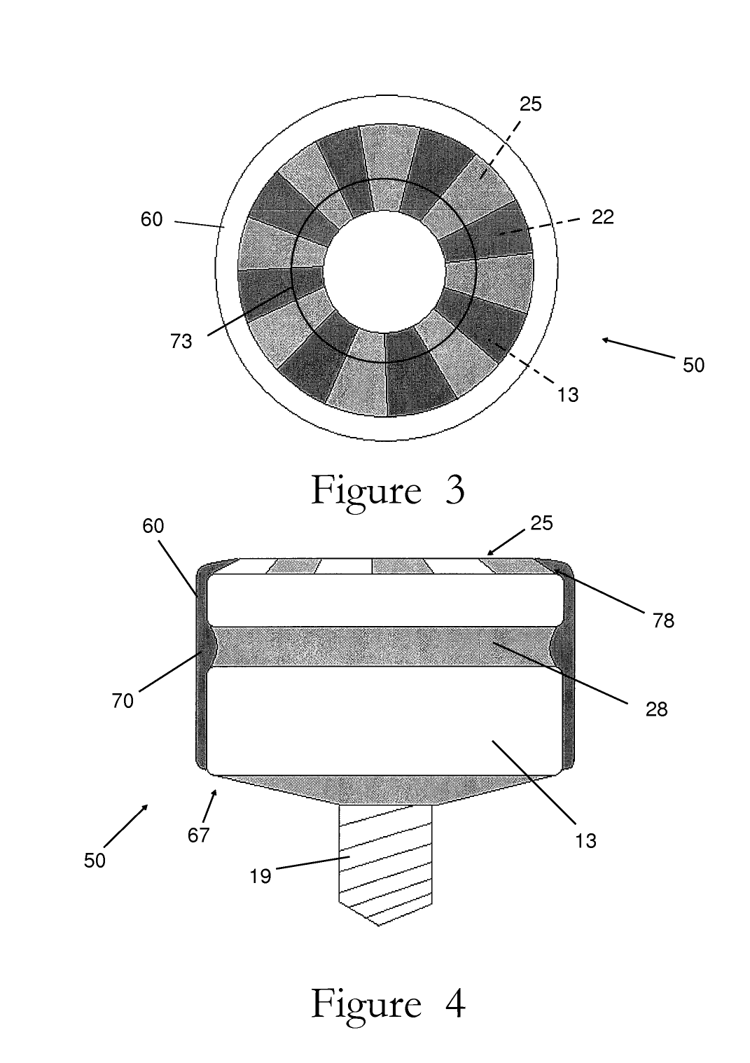

FIGS. 3 and 4 show a customizable healing abutment, indicated generally as 50, according to the present invention. The customizable healing abutment 50 includes a central abutment piece 13 and a customizable gingival emergence profile shell 60. As described above, the abutment piece 13 is generally cylindrical in shape and adapted for attachment to an oral implant, typically by using a screw or threaded post 19. The top surface 22 of the abutment piece 13 includes a plurality of facets 25 that create a multi-faceted occlusal stop/lock. In some embodiments, a plurality of teeth or other features that will lock the orientation of the device can be used. A detent 28 is formed around the periphery of the cylindrical wall of the abutment piece 13.

The customizable profile shell 60 is generally cylindrical in shape. In a preferred embodiment, the profile shell 60 should be made of a compatible material having sufficient strength and flexibility. Preferably, material can be added to or removed from the customizable profile shell 60 to enable a clinician to determine the final shape of the customizable profile shell 60. The customizable profile shell 60 includes a hollow, cylindrical central area 67 and is adapted to be removably placed over the abutment piece 13. A lip 70 is formed on an inner wall of the customizable profile shell 60. The lip 70 is sized and configured to engage the detent 28 of the abutment piece 13 in a “snap” engagement when the customizable shell 60 is fit into place on the abutment piece 13. A top rim 73 of the shell 16 overlaps at least a portion of the top surface 22 of the abutment piece 13. The inner surface 78 of the top rim 73 includes a plurality of facets that are configured to match the facets 25 of the top surface 22 of the abutment piece 13 in order to create a multi-faceted occlusal stop/lock.

The customizable shell 60 is used in a similar manner as the pre-made shell 16. First, the abutment piece 13 will be secured to an underlying dental implant by the screw or threaded post 19. A customizable profile shell 60 is then snapped onto the abutment piece 13. In a preferred embodiment, a clinician can customize the shape of the shell 60 to create gingival contours surrounding an implant prosthesis, and which imitate the gingival contours, before or after the customizable shell 60 is put on the abutment piece 13. The plurality of multiple facets 25 on the top surface 22 of the abutment piece 13 when matched with the facets on the inner surface 78 of the top rim 73 prevents rotation of the customizable gingival profile shell 60.

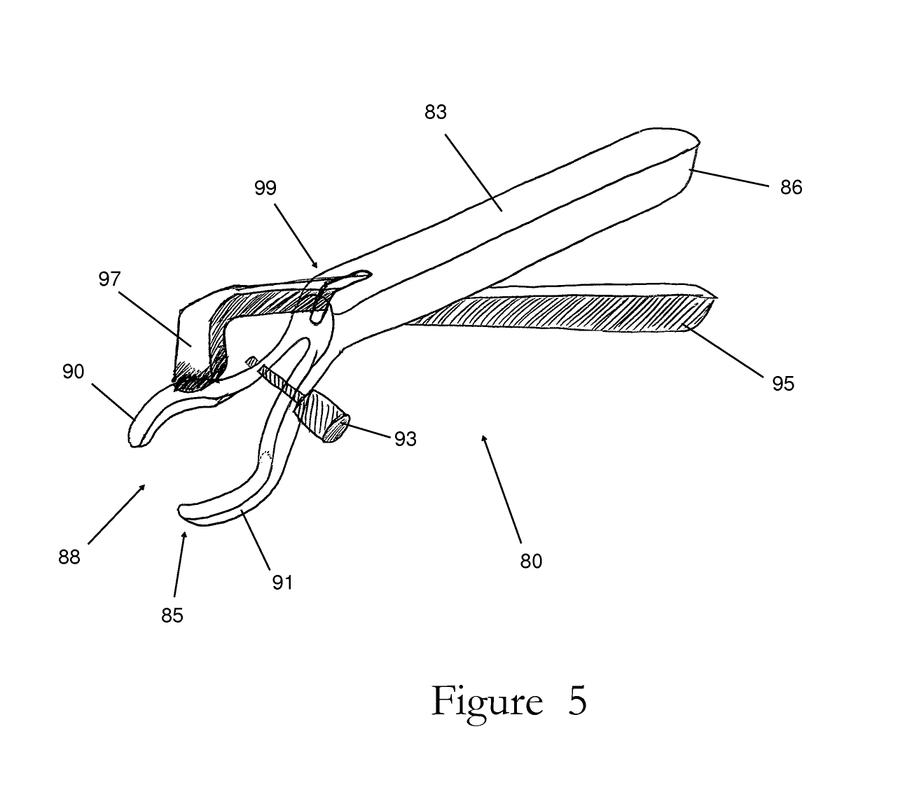

FIG. 5 shows a shell removal tool, indicated generally as 80, according to the present invention. The removal tool 80 includes a handle 83 having a first end 85 and a second end 86. The first end 85 is divided into a fork 88 having two prongs 90, 91. The prongs 90, 91 have a generally curvilinear shape, sized and configured to fit around a gingival emergence profile shell 16, 60. In a preferred embodiment, the fork 88 includes a locking device 93 adapted to hold the prongs 90, 91 in engaged relationship with an emergence profile shell 16, 60. Preferably, the removal tool 80 also includes a lever arm 95 hingedly connected adjacent the fork 88 on the first end of the handle 83. On one end of the lever arm 95 is provided a thumb 97 that is adapted to engage the top of abutment piece 13. The thumb 97 extends substantially perpendicular to the lever arm 95. In a preferred embodiment, the thumb 97 helps provide mechanical advantage to snap the emergence profile shell 16, 60 off the abutment piece 13. In some embodiments, movement of the lever arm 95 with respect to the handle 83 may be resisted by friction at the hinged joint 99. In other embodiments, the removal tool 80 may include a spring or similar device (not shown).

To use the shell removal tool 80, an emergence profile shell 16, 60 is grasped between prongs 90, 91 and held by tightening the locking device 93. The lever arm 95 is then moved to bring the thumb 97 in contact with the top surface 22 of the abutment piece 13. The remaining end of the lever arm 95 is brought substantially parallel to the handle 83 thereby pressing the thumb 97 onto the top surface 22 of the abutment piece 13 causing the profile shell 16, 60 to “pop” off.

### INDUSTRIAL APPLICABILITY

The present invention is applicable to providing a snap-on two-piece emergence profile healing abutment. The invention discloses a central healing abutment piece having multiple surface facets and an interchangeable outer gingival emergence profile shell piece.

What is claimed is:

1. A gingival emergence profile, comprising:
an abutment piece having a generally cylindrical body with a threaded post on an end of said elongate body, said body having a detent around the periphery of the body and a plurality of facets on a top surface thereof; and
a shell for providing gingival support, comprising a shell body having a distal end and a proximal end with a sidewall extending therebetween defining a sleeve interior, said sidewall having an interior wall with a lip formed thereon, said body having a rim on the proximal end, wherein said rim overlaps at least a portion of the top surface of the abutment piece and includes a plurality of facets on an inner surface thereof.

2. The emergence profile according to claim 1, wherein the shell is interchangeable on the abutment piece.

3. The emergence profile according to claim 1, wherein a circumference of the exterior surface of the shell body adjacent the proximal end is greater than a circumference of the exterior surface of the shell body adjacent the distal end.

4. The emergence profile according to claim 3, wherein the shell body is tapered from the proximal end to the distal end.

5. The emergence profile according to claim 1, wherein the distal end of the shell body defines an opening.

6. The emergence profile according to claim 1, wherein the shell interior of the sleeve body is adapted to at least partially receive the abutment piece therein.

7. The emergence profile according to claim 1, wherein the lip is sized and configured to engage the detent around the periphery of the cylindrical body of the abutment piece.

8. The emergence profile according to claim 1, wherein the abutment piece has a distal end and a proximal end with a sidewall extending therebetween defining a cylindrical body.

9. The emergence profile according to claim 8, wherein the distal end of the cylindrical body is adapted to engage a receiving socket of a dental implant body.

10. The emergence profile according to claim 9, wherein the dental implant body is secured into a dental bone socket of a patient.

11. The emergence profile according to claim 8, wherein the proximal end of the cylindrical body comprises a plurality of facets that create a multi-faceted occlusal stop/lock.

12. The emergence profile according to claim 1, wherein the shell is at least partially rotatable about a portion of the abutment piece.

13. A method of providing gingival support during a dental implant procedure, comprising the steps of:
securing a distal end of a dental implant body into a dental bone socket of a patient;
engaging a distal end of a cylindrical abutment piece with a receiving socket of the dental implant body at a proximal end of the dental implant body, wherein the cylindrical abutment piece comprises a detent circumscribing a periphery thereof;
placing a shell body having a distal end and a proximal end with a sidewall extending therebetween defining a sleeve interior over the cylindrical abutment piece so that the interior of the sleeve body at least partially receives the abutment piece therein, said shell comprising a lip formed thereon; and
affixing the shell in a desired orientation about the abutment piece, wherein the lip engages the detent.

14. The method according to claim 13, further comprising the steps of:
selecting a pre-made emergence profile shell.

15. The method according to claim 14, wherein the shell is aligned with a surrounding gum line and teeth before affixation.

16. The method according to claim 13, further comprising the steps of:
customizing the shape of the emergence profile shell.

17. The method according to claim 13, wherein the shell is at least partially rotatable about a portion of the abutment piece before affixation.
